(19) United States
(12) Patent Application Publication
(10) Pub. No.: US 2026/0120884 A1
(43) Pub. Date: Apr. 30, 2026

(54) BIOLOGICAL TISSUE INFORMATION PROCESSING APPARATUS, BIOLOGICAL TISSUE INFORMATION PROCESSING METHOD, AND STORAGE MEDIUM

(71) Applicant: CANON MEDICAL SYSTEMS CORPORATION, Otawara-shi (JP)

(72) Inventors: Kazuki UTSUNOMIYA, Nasushiobara (JP); Fuminori FUJITA, Nasushiobara (JP); Atsuko SUGIYAMA, Ota (JP)

(73) Assignee: CANON MEDICAL SYSTEMS CORPORATION, Otawara-shi (JP)

(21) Appl. No.: 19/314,133

(22) Filed: Aug. 29, 2025

(30) Foreign Application Priority Data
Sep. 30, 2024 (JP) 2024-170092

(51) Int. Cl. G16H 50/30; A61B 5/00

(52) U.S. Cl. CPC G16H 50/30; A61B 5/0077; A61B 5/4842; A61B 5/721; A61B 5/7267; A61B 5/7278; A61B 5/7282; A61B 5/742; A61B 2576/00

(57) ABSTRACT

A biological tissue information processing apparatus according to an exemplary embodiment includes an acquisition unit, a comparison unit, a tissue change estimation unit, and a display control unit. The acquisition unit acquires tissue information indicating a state of tissue related to a disease in a body of a subject, and body surface information indicating a state of a body surface of the subject. The comparison unit compares the tissue information with the body surface information and calculates a result of a comparison between the tissue information and the body surface information. The tissue change estimation unit estimates a change in characteristics of the tissue based on a temporal change of the result of the comparison. The display control unit controls a terminal to display an estimation result of the change in the characteristics of the tissue.

### CROSS-REFERENCE TO RELATED APPLICATIONS

This application is based upon and claims the benefit of priority from Japanese Patent Application No. 2024-170092, filed Sep. 30, 2024, the entire contents of which are incorporated herein by reference.

### FIELD

Embodiments described herein relate generally to a biological tissue information processing apparatus, a biological tissue information processing method, and a storage medium.

### BACKGROUND

Characteristics (e.g., hardness, accumulation of fluid) of lesional tissue (or tissue surrounding a lesion) of a subject may change (e.g., become harder, become softer, or accumulate fluid) in association with the onset, progression, or recurrence of a disease in the subject. For this reason, it is important for the treatment of some diseases in the subject to detect a change in the characteristics of tissue of a lesional region in the subject at an early stage.

However, in a conventional procedure in which a disease is detected by X-ray computed tomography (CT) imaging or the like following radiography when a symptom appears in the subject, there is an issue that it is difficult to detect a disease or the like of the subject before a symptom appears in the subject. For example, to improve the quality of life (QOL) of the subject, in various follow-ups including a postoperative follow-up, it is desirable that various symptoms be detected at an early stage before the symptoms appear.

### BRIEF DESCRIPTION OF THE DRAWINGS

FIG. 1 is a block diagram illustrating a configuration example of a biological tissue information processing system including a biological tissue information processing apparatus according to an exemplary embodiment;

FIG. 2 is a view illustrating an example of a positional relationship between an intracorporeal device and an extracorporeal device according to the exemplary embodiment;

FIG. 3 is a table illustrating examples of tissue information and body surface information according to the exemplary embodiment;

FIG. 4 is a table illustrating an example of a result of matching between the tissue information and the body surface information illustrated in FIG. 3 according to the exemplary embodiment;

FIG. 5 is a graph illustrating an example of a difference based on an acquisition date and time according to the exemplary embodiment;

FIG. 6 is a graph illustrating an example of the difference relative to the acquisition date and time, and a trend value according to the exemplary embodiment;

FIG. 7 is a graph illustrating an example of the difference in a case where a disease condition of a subject is pleural effusion, and a trend value according to the exemplary embodiment;

FIG. 8 is a diagram illustrating an example of an estimation result displayed on a display of a terminal according to the exemplary embodiment;

FIG. 9 is a diagram illustrating examples of a graph indicating the size of a tumor over time according to a comparative example, and a graph indicating the size of the tumor over time according to the exemplary embodiment;

FIG. 10 is a diagram illustrating an example of an estimation result regarding a tumor displayed on the display of the terminal according to the exemplary embodiment;

FIG. 11 is a diagram illustrating an example of an estimation result regarding pleural effusion displayed on the display of the terminal according to the exemplary embodiment;

FIG. 12 is a flowchart illustrating an example of a procedure of characteristic change estimation processing according to the exemplary embodiment; and

FIG. 13 is a diagram illustrating an example of an estimation result displayed on a display of an electrocardiogram monitor in a case where the electrocardiogram monitor is used as a body motion detection device according to a second application example of the exemplary embodiment.

### DETAILED DESCRIPTION

A biological tissue information processing apparatus according to an exemplary embodiment includes an acquisition unit, a comparison unit, a tissue change estimation unit, and a display control unit. The acquisition unit acquires tissue information indicating a state of tissue related to a disease in a body of a subject, and body surface information indicating a state of a body surface of the subject. The comparison unit compares the tissue information with the body surface information and calculates a result of a comparison between the tissue information and the body surface information. The tissue change estimation unit estimates a change in characteristics of the tissue based on a temporal change of the result of the comparison. The display control unit controls a terminal to display an estimation result of the change in the characteristics of the tissue.

Various Embodiments will be described hereinafter with reference to the accompanying drawings.

In the following exemplary embodiments, assume that components denoted by the same reference numerals perform similar operations, and redundant description is omitted as appropriate.

### Exemplary Embodiment

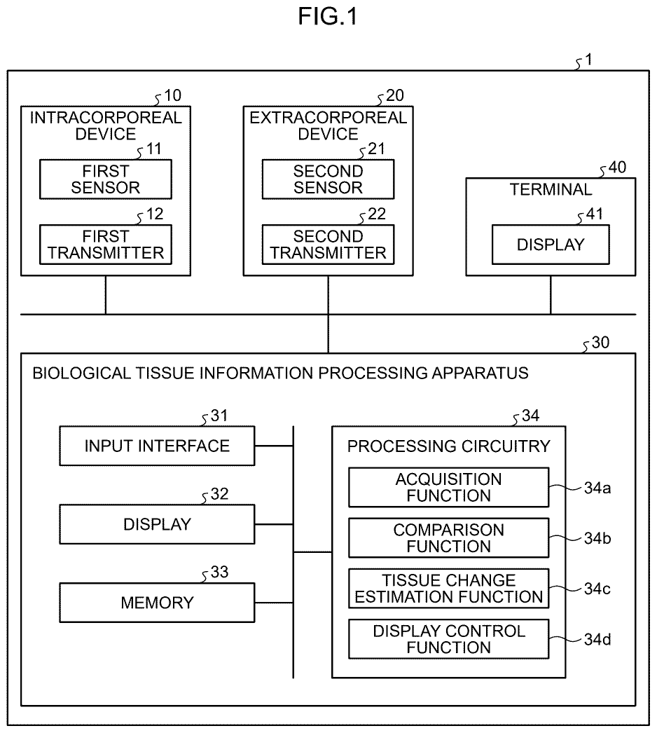

FIG. 1 is a block diagram illustrating a configuration example of a biological tissue information processing system 1 including a biological tissue information processing apparatus 30 according to an exemplary embodiment. As illustrated in FIG. 1, the biological tissue information processing system 1 according to the exemplary embodiment includes an intracorporeal device 10, an extracorporeal device 20, the biological tissue information processing apparatus 30, and a terminal 40. The intracorporeal device 10, the extracorporeal device 20, and the terminal 40 are connected to the biological tissue information processing apparatus 30 by wired connection or wireless connection via the Internet and/or a network such as an intranet. A connection destination of the biological tissue information processing apparatus 30 is not limited to the intracorporeal device 10 and the extracorporeal device 20 described above, but may also be another server and/or another database, and the like.

The intracorporeal device 10 is a device that is located inside the body of a subject and corresponds to, for example, an implantable wireless device. The intracorporeal device 10 is located in a region corresponding to a disease (hereinafter referred to as a lesional region) in the subject. For example, the intracorporeal device 10 is located in an area associated with tissue related to a disease (hereinafter referred to as lesional tissue) in the body of the subject with regard to the lesional region. The area associated with the lesional tissue corresponds to, for example, the lesional tissue itself or tissue surrounding the lesional tissue (hereinafter referred to as surrounding tissue). The intracorporeal device 10 is located in the lesional tissue when a surgical treatment, puncture, biopsy, or the like is implemented on the lesional tissue.

As the intracorporeal device 10, for example, an existing implantable device such as a pacemaker may be used. The intracorporeal device 10 may be covered with, for example, a film attached to the surface of damaged tissue, a biomimetic surface with a surfactant polymer, or the like. With this configuration, adhesion between the subject and the intracorporeal device 10 can be reduced inside the body of the subject, and an adverse effect of the intracorporeal device 10 on the subject can be reduced.

In a case where the lesion involves cancer, the lesional tissue corresponds to an area of cancerous tissue, and the intracorporeal device 10 is located in the area indicating cancer. In a case where the lesion is osteoporosis, the lesional region corresponds to a bone portion with a lower bone density, and the intracorporeal device 10 is located in the bone portion with the lower bone density. In a case where the lesion involves pleural effusion accumulated in a thoracic cavity (between the lungs and the chest wall), cardiac tamponade, malignant pericardial effusion, or the like, the lesional region corresponds to the thoracic region, and the intracorporeal device 10 is located in the thoracic region. In a case where the lesion involves ascites such as malignant ascites, the lesional region corresponds to the abdominal region, and the intracorporeal device 10 is located in the abdominal region.

In a case where the lesion involves dementia or chronic subdural hematoma, the lesional region corresponds to the brain, and the intracorporeal device 10 is located in the brain. In a case where the lesion involves obesity, the lesional region corresponds to an area with a high fat concentration, and the intracorporeal device 10 is located in the area with a high fat concentration. In a case where the lesion involves liver cirrhosis, the lesional region corresponds to the liver, and the intracorporeal device 10 is located in the liver. In a case where the lesion involves muscle, the lesional region corresponds to the muscle, and the intracorporeal device 10 is located in the muscle.

The intracorporeal device 10 includes a first sensor 11 and a first transmitter 12. The first sensor 11 detects tissue information indicating a state of tissue related to a disease in the body of the subject. The first sensor 11 corresponds to, for example, a sensor that detects mechanical characteristics of lesional tissue or surrounding tissue. Specifically, the first sensor 11 is implemented as an acceleration sensor that detects the acceleration of the lesional tissue or the surrounding tissue, a temperature sensor that detects the temperature of the lesional tissue or the surrounding tissue, a pressure sensor that detects the pressure of the lesional tissue or the surrounding tissue, or the like. For the sake of specific description, the first sensor 11 is described as an acceleration sensor. In this case, the tissue information corresponds to the acceleration (intracorporeal acceleration) of the lesional tissue or the surrounding tissue.

The first transmitter 12 transmits the tissue information including a tissue information acquisition date and time to the biological tissue information processing apparatus 30. Since a known wireless communication technique such as proximity communication can be applied to the first transmitter 12, the description thereof is omitted. In a case where the intracorporeal device 10 and the biological tissue information processing apparatus 30 can be connected with a wire, the first transmitter 12 transmits the tissue information to the biological tissue information processing apparatus 30 via a cable that electrically connects the intracorporeal device 10 and the biological tissue information processing apparatus 30.

While the example of the intracorporeal device 10 has been described above, the intracorporeal device 10 is not limited to the above-described example. For example, the intracorporeal device 10 may be implemented using a magnetic material. The magnetic material corresponds to, for example, a specific marker located inside the body of a subject P at a location of a surgical treatment or puncture. The specific marker corresponds to, for example, a metallic marker or the like located in a breast of the subject P in case a lesion disappears during chemotherapy given before surgery on breast cancer. In this case, the metallic marker may be located in the breast of the subject P in breast puncture. Acquisition of the tissue information in this case will be described in the description of an acquisition function 34a.

The extracorporeal device 20 is a device that is located outside the body of the subject P and is attached to, for example, the body surface of the subject P. For example, the extracorporeal device 20 is attached to the body surface of the subject P at a position the closest to the intracorporeal device 10 or the lesional region. The extracorporeal device 20 is not limited to the device of a type that is constantly attached to the body surface of the subject P when various kinds of processing according to the present exemplary embodiment are executed, but instead may be implemented as, for example, a device installed on a chair for use by the subject P while seated, or a device separately installed for use during the subject P's sleep.

The extracorporeal device 20 is not limited to a device of the type that is attached to the subject P, but instead may be implemented as a contactless device relative to the subject P, such as a device capable of capturing an image of the subject P (e.g., an optical camera), or a device that transmits and receives radio waves via a wireless local area network (LAN) or a millimeter-wave radar. Further, the extracorporeal device 20 may be incorporated in, for example, a sensor (an electrocardiographic sensor or the like) that measures an electrocardiogram of the subject P. For the sake of specific description, the extracorporeal device 20 is described as a device that is attached to the subject P.

The extracorporeal device 20 includes a second sensor 21 and a second transmitter 22. The second sensor 21 detects body surface information indicating a state of the body surface of the subject P. The second sensor 21 corresponds to, for example, a sensor that detects mechanical characteristics of the body surface of the subject P. Specifically, the second sensor 21 is implemented as an acceleration sensor that detects the acceleration (extracorporeal acceleration) of the body surface of the subject P, a temperature sensor that detects the temperature of the body surface of the subject P, a pressure sensor that detects the pressure of the body surface of the subject P, or the like. For the sake of specific description, the second sensor 21 is described as an acceleration sensor. In this case, the body surface information corresponds to the acceleration of the body surface of the subject P.

The second transmitter 22 transmits the body surface information including a body surface information acquisition date and time to the biological tissue information processing apparatus 30. Since a known wireless communication technique can be applied to the second transmitter 22, the description thereof is omitted. In a case where the extracorporeal device 20 and the biological tissue information processing apparatus 30 can be connected with a wire, the second transmitter 22 transmits the body surface information to the biological tissue information processing apparatus 30 via a cable that electrically connects the extracorporeal device 20 and the biological tissue information processing apparatus 30.

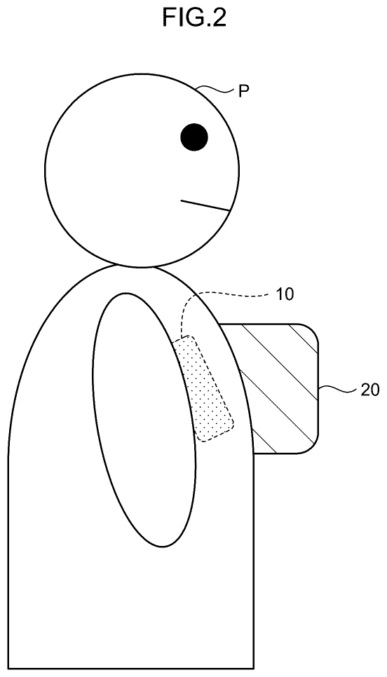

FIG. 2 is a view illustrating an example of a positional relationship between the intracorporeal device 10 and the extracorporeal device 20. In FIG. 2, the intracorporeal device 10 is located in the chest in the body of the subject P. In this case, as illustrated in FIG. 2, the extracorporeal device 20 is located on the front side of the intracorporeal device 10, i.e., on the body surface of the subject P so as to face the intracorporeal device 10 and at a position the closest to the intracorporeal device 10.

In a case where the extracorporeal device 20 is constantly attached to the subject P, the intracorporeal device 10 and the extracorporeal device 20 may include a schedule timer. In this case, the intracorporeal device 10 and the extracorporeal device 20 may be operated during a preliminarily planned (set) time based on the schedule timer. The preliminarily set time may be, for example, 2:00 to 3:00, 8:00 to 9:00, 16:00 to 17:00, or the like.

The terminal 40 includes a display 41, an input interface (not illustrated), processing circuitry (not illustrated), and a memory (not illustrated). The terminal 40 corresponds to a client apparatus or a viewer apparatus in an electronic health record system and/or a biological tissue information system, and/or a smartphone or the like of the subject P on which a health support application, a sleep analysis application, or the like is installed.

The processing circuitry in the terminal 40 is implemented as, for example, a processor. The term “processor” refers to circuitry such as a central processing unit (CPU), a graphics processing unit (GPU), an application-specific integrated circuit (ASIC), or a programmable logic device (e.g., a simple programmable logic device (SPLD), a complex programmable logic device (CPLD), and a field-programmable gate array (FPGA)). The processing circuitry in the terminal 40 controls the entire terminal 40 based on programs stored in the memory (not illustrated).

The memory in the terminal 40 stores data to be displayed by a display control function 34d and other data. The memory is implemented as, for example, a semiconductor memory element such as a random access memory (RAN) or a flash memory, a hard disk, an optical disk, or the like. The memory in the terminal 40 also stores programs for each circuitry included in the terminal 40 to implement functions of the circuitry. The memory is an example of a terminal storage unit.

In a case where the terminal 40 is a client apparatus, the input interface is implemented as, for example, a trackball, switches, buttons, a mouse, or a keyboard for providing various instructions, various settings, and the like, a touch pad on which an input operation is performed by touching an operation surface thereof, a touch screen in which a display screen and a touch pad are integrally formed, contactless input circuitry using an optical sensor, voice input circuitry (e.g., a microphone), or the like. In a case where the terminal 40 is a smartphone, the input interface is implemented as switches, buttons, a touch pad, a touch screen, voice input circuitry, or the like for providing various instructions, various settings, and the like. The input interface converts an input operation received from a user into an electric signal and outputs the electric signal to the processing circuitry in the terminal 40.

The input interface in the terminal 40 is not limited to those including physical operation members such as a mouse and a keyboard. An example of the input interface may be electric signal processing circuitry that receives an electric signal corresponding to an input operation from an external input device provided separately from the terminal 40, and outputs the electric signal to the processing circuitry in the terminal 40. The input interface is an example of an input unit, and may also be referred to as an operation unit.

The display 41 displays various kinds of information under the control of the processing circuitry in the terminal 40 and/or the control of the display control function 34d. For example, the display 41 displays a graphical user interface (GUI) and the like for receiving instructions from the user. For example, the display 41 is a liquid crystal display, a cathode-ray tube (CRT) display, an organic electro-luminescence (EL) display, or the like. The display 41 is an example of a display unit. The display 41 displays an estimation result estimated by a tissue change estimation function 34c, and the like under the control of the display control function 34d. Contents to be displayed on the display 41 will be described below.

The biological tissue information processing apparatus 30 is implemented as, for example, a server apparatus. The biological tissue information processing apparatus 30 may also be implemented as various medical servers such as a medical image management system, namely, Picture Archiving and Communication System (PACS). As illustrated in FIG. 1, the biological tissue information processing apparatus 30 includes an input interface 31, a display 32, a memory 33, and processing circuitry 34.

The input interface 31 is implemented as, for example, a trackball, switches, buttons, a mouse, and a keyboard for providing various instructions, various settings, and the like, a touch pad on which an input operation is performed by touching an operation surface thereof, a touch screen in which a display screen and a touch pad are integrally formed, contactless input circuitry using an optical sensor, voice input circuitry (e.g., a microphone), or the like. The input interface 31 converts an input operation received from the user into an electric signal and outputs the electric signal to the processing circuitry 34.

The input interface 31 is not limited to those including physical operation members such as a mouse and a keyboard. An example of the input interface 31 may be electric signal processing circuitry that receives an electric signal corresponding to an input operation from an external input device provided separately from the biological tissue information processing apparatus 30, and outputs the electric signal to the processing circuitry 34. The input interface 31 is an example of the input unit, and may also be referred to as the operation unit.

The display 32 displays various kinds of information under the control of a display control function 34d. For example, the display 32 displays a GUI for receiving instructions from the user, an estimation result estimated by the tissue change estimation function 34c, and the like. For example, the display 32 is a liquid crystal display or an organic EL display. The display 32 is an example of the display unit.

The memory 33 is implemented as, for example, a semiconductor memory element such as a RAM or a flash memory, a hard disk, an optical disk, or the like. For example, the memory 33 stores various kinds of data generated through various kinds of processing performed by the processing circuitry 34 to be described below. The memory 33 also stores an estimation result estimated by the tissue change estimation function 34c, and the like. The memory 33 also stores programs for each circuitry included in the biological tissue information processing apparatus 30 to implement the functions of the circuitry. The memory 33 is an example of a storage unit.

The processing circuitry 34 may be implemented as a processor. The processing circuitry 34 controls the overall operation of the biological tissue information processing apparatus 30 by executing the acquisition function 34a, a comparison function 34b, the tissue change estimation function 34c, and the display control function 34d. The processing circuitry 34 that implements the acquisition function 34a corresponds to an acquisition unit. The processing circuitry 34 that implements the comparison function 34b corresponds to a comparison unit. The processing circuitry 34 that implements the tissue change estimation function 34c corresponds to a tissue change estimation unit. The processing circuitry 34 that implements the display control function 34d corresponds to a display control unit.

The processing circuitry 34 reads out a program corresponding to the acquisition function 34a from the memory 33 and executes the program. Thus, the acquisition function 34a acquires tissue information indicating a state of tissue related to a disease in the body of the subject P, and body surface information indicating a state of a body surface of the subject P. For example, the acquisition function 34a acquires the tissue information from the intracorporeal device 10. The acquisition function 34a causes the memory 33 to store the acquired tissue information. In a case where the intracorporeal device 10 is formed of a magnetic material, the acquisition function 34a includes, for example, a function capable of detecting a motion of the magnetic material. In this case, the acquisition function 34a functions as a magnetic transmitter capable of detecting a position of the magnetic material. The acquisition function 34a detects the position of the magnetic material over time, thereby acquiring an acceleration indicating a motion of the magnetic material in the body as an intracorporeal acceleration in the tissue information.

The acquisition function 34a acquires the body surface information from the extracorporeal device 20. The acquisition function 34a causes the memory 33 to store the acquired body surface information. As a modified example of the present exemplary embodiment, the acquisition function 34a may input tissue information to a trained model and acquire body surface information from the trained model. In this case, the trained model is trained in advance and is stored in the memory 33. The acquisition function 34a may acquire the body surface information from the tissue information using statistical analysis processing in place of a trained model. In this case, a program that implements the statistical analysis processing is preliminarily stored in the memory 33.

In a case where the extracorporeal device 20 is implemented as a contactless device relative to the subject P, the acquisition function 34a may acquire the body surface information based on an output from the contactless device. For example, in a case where an optical camera is applied as the contactless device, the acquisition function 34a executes image analyses on a plurality of images acquired in time series by the optical camera, thereby acquiring the body surface information including the acceleration on the body surface. Since known image processing methods such as position alignment processing and differential processing can be applied as the image analyses, the description thereof is omitted.

In a case where a wireless LAN or a millimeter-wave radar (e.g., Light Detection and Ranging (LiDAR)) is applied as the contactless device, the acquisition function 34a executes various kinds of data processing on data acquired by the wireless LAN or the millimeter-wave radar, thereby acquiring the body surface information including the acceleration on the body surface. Since known data processing methods can be applied as the data processing, the description thereof is omitted.

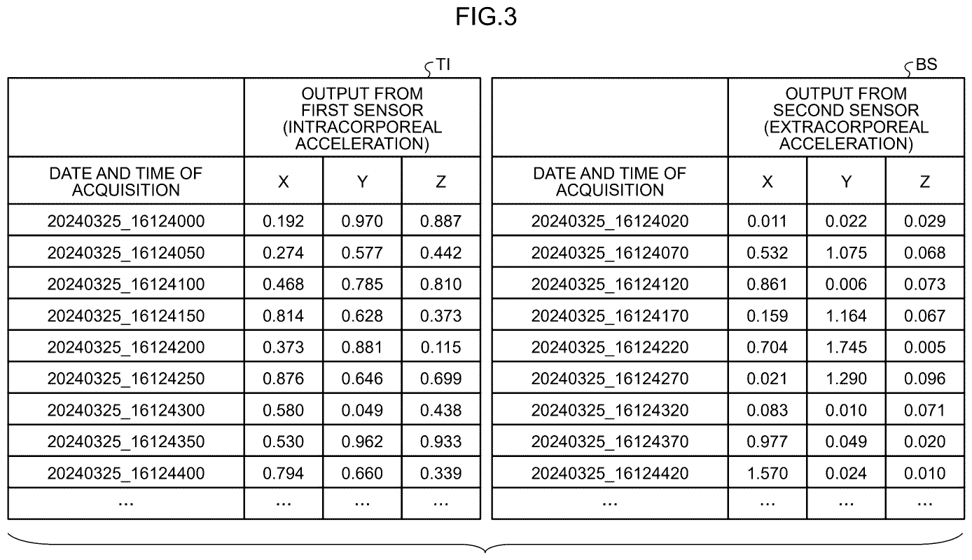

FIG. 3 is a table illustrating examples of tissue information TI and body surface information BS. In a case where the first sensor 11 and the second sensor 21 are each implemented as an acceleration sensor (triaxial acceleration sensor) corresponding to three axes (X-axis, Y-axis, and Z-axis) that are orthogonal to each other, an output (intracorporeal acceleration in tissue information) from the first sensor 11 and an output (extracorporeal acceleration in body surface information) from the second sensor 21 include accelerations corresponding to the three axes (X-axis, Y-axis, and Z-axis), respectively, and an acquisition date and time of each acceleration.

In a case where the extracorporeal device 20 is constantly attached to the subject P and the intracorporeal device 10 and the extracorporeal device 20 each include a schedule timer, the acquisition function 34a acquire tissue information and body surface information depending on operation of each of the intracorporeal device 10 and the extracorporeal device 20 at a planned time. While FIG. 3 illustrates an example where the tissue information TI and the body surface information BS are acquired at constant time intervals, the present exemplary embodiment is not limited thereto. The tissue information TI and the body surface information BS may be acquired based on signals from the outside of the body of the subject P. The contents of processing for acquiring the tissue information TI and the body surface information BS based on signals from the outside of the body of the subject P will be described as an application example of the present exemplary embodiment.

The processing circuitry 34 reads out a program corresponding to the comparison function 34b from the memory 33 and executes the program. Thus, the comparison function 34b compares the tissue information with the body surface information. The comparison function 34b calculates a comparison result obtained by the comparison. For example, the comparison function 34b performs matching of the acquisition date and time of the tissue information and the acquisition date and time of the body surface information. Accordingly, the comparison function 34b cross-references the acquisition date and time of the tissue information and the acquisition date and time of the body surface information, thereby identifying the corresponding acceleration, for example, in units of one-tenth of a second.

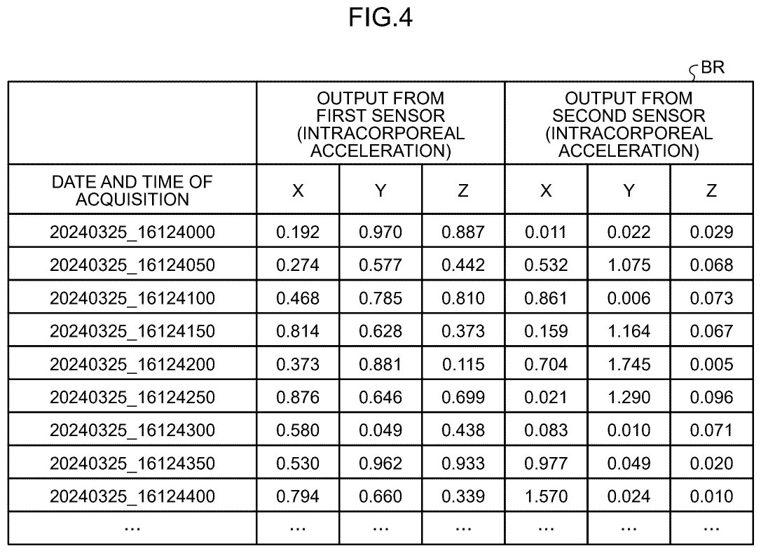

FIG. 4 is a table illustrating an example of a matching result BR between the tissue information TI and the body surface information BS illustrated in FIG. 3. The matching result BR illustrated in FIG. 4 indicates the intracorporeal acceleration and the extracorporeal acceleration obtained after the matching in units of one-tenth of a second. The comparison function 34b causes the memory 33 to store the matching result BR.

The comparison function 34b calculates a difference (also referred to as differential data) between the tissue information TI and the body surface information BS as the comparison result for every acquisition date and time using the tissue information TI and the body surface information BS obtained after the matching. A formula for calculating a difference Δ for every acquisition date and time after matching is given as, for example, the following Formula (1) or (2). The formula for calculating the difference Δ is not limited to Formula (1) and Formula (2), and may be expressed by another formula as long as the difference between the acceleration of the tissue and the acceleration on the body surface after matching can be expressed.

$$Difference Δ = \vert ( {X}_{intra} - {X}_{extra} ) + ( {Y}_{intra} - {Y}_{extra} ) + ( {Z}_{intra} - {Z}_{extra} ) \vert \qquad ( 1 )$$

$$Difference Δ = \{ ( {X}_{intra} - {X}_{extra} ) ( {Y}_{intra} - {Y}_{extra} ) ( {Z}_{intra} - {Z}_{extra} ) \} \hat{} ( 1 / 2 ) \qquad ( 2 )$$

In Formula (1) and Formula (2), the suffix “intra” represents a portion (tissue) in the body of the subject P, and the suffix “extra” represents a portion (body surface) outside the body of the subject P. Further, in Formula (1) and Formula (2), Xᵢₙₜᵣₐ represents an X-axis acceleration on the tissue in the body of the subject P, Yᵢₙₜᵣₐ represents a Y-axis acceleration on the tissue in the body of the subject P, and Zᵢₙₜᵣₐ represents a Z-axis acceleration on the tissue in the body of the subject P. In Formula (1) and Formula (2), Xₑₓₜᵣₐ represents an X-axis acceleration on the body surface of the subject P, Yₑₓₜᵣₐ represents a Y-axis acceleration on the body surface of the subject P, and Zₑₓₜᵣₐ represents a Z-axis acceleration on the body surface of the subject P. As represented by Formula (1) and Formula (2), the difference Δ is based on the extracorporeal acceleration.

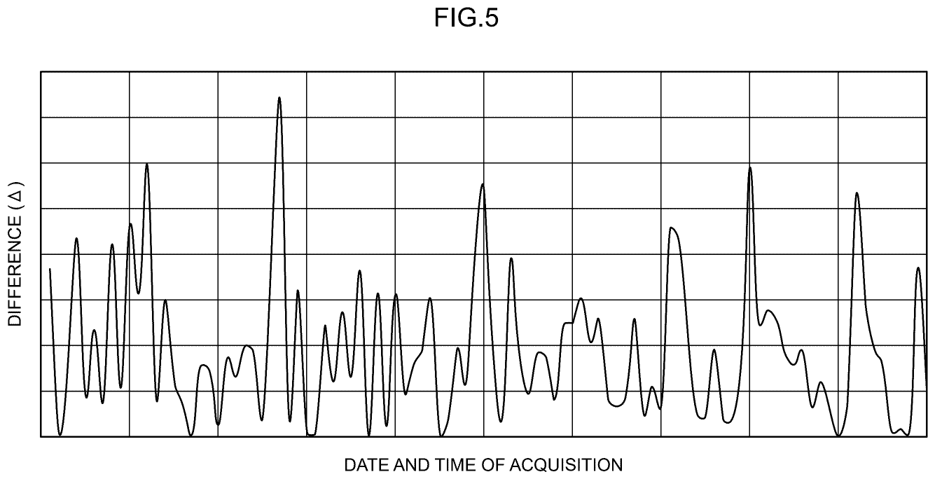

The comparison function 34b associates the difference Δ calculated for each acquisition date and time with the acquisition date and time, and causes the memory 33 to store the difference Δ associated therewith as the comparison result. FIG. 5 is a graph illustrating an example of the difference Δ with regard to the acquisition date and time. The vertical axis illustrated in FIG. 5 represents the magnitude of the difference Δ. The horizontal axis illustrated in FIG. 5 represents the acquisition date and time (e.g., in the order of seconds). As illustrated in FIG. 5, the magnitude of the difference Δ fluctuates significantly. When the tissue surrounding the intracorporeal device 10 is soft, a discrepancy occurs between a vibration pattern of the acceleration detected by the intracorporeal device 10 and a vibration pattern of the acceleration detected by the extracorporeal device 20 (the difference between the devices is large). Specifically, when the tissue surrounding the intracorporeal device 10 is soft, or when a larger amount of liquid such as fluid is present around the tissue, the difference between the acceleration of the tissue and the acceleration on the body surface increases.

On the other hand, when the tissue surrounding the intracorporeal device 10 is hard, the intracorporeal device 10 and the extracorporeal device 20 vibrate in the same manner (the difference between the devices is small). Specifically, when the tissue surrounding the intracorporeal device 10 is hard, or when only a smaller amount of liquid such as fluid is present around the tissue, the difference between the acceleration of the tissue and the acceleration on the body surface decreases.

The processing circuitry 34 causes the tissue change estimation function 34c to estimate a change in characteristics of the tissue based on a temporal change of the comparison result. The characteristics of the tissue include, for example, the hardness of the tissue in association with the location of the intracorporeal device 10, and accumulation of fluid or air around the tissue. For example, the tissue change estimation function 34c applies a moving average of the difference Δ with regard to the acquisition date and time, thereby calculating a trend of the difference Δ (hereinafter referred to as a trend value).

Specifically, the tissue change estimation function 34c calculates, as a trend value, an average of differences A over a predetermined previous period from a reference acquisition date and time based on a plurality of acquisition dates and times serving as references. The predetermined previous period is, for example, 10 seconds. Thus, the tissue change estimation function 34c calculates the average of the differences A over the immediately previous period, thereby calculating the trend value for each of the plurality of acquisition dates and times. The calculation of the trend value makes it possible to reduce the fluctuation (distribution) of the difference Δ.

Next, the tissue change estimation function 34c compares the trend value with a predetermined threshold. The predetermined threshold is preliminarily set as a table in accordance with, for example, a region related to a disease condition of the subject P, a location where the intracorporeal device 10 is placed (implanted), and a disease name, an organ, and an inspection order of the subject P, and is stored in the memory 33. The predetermined threshold may be set using a trend value obtained at a timing when the disease condition of the subject P has previously deteriorated. The tissue change estimation function 34c causes the memory 33 to store the result of comparison between the trend value and the predetermined threshold as an estimation result of a change in the characteristics of the tissue.

If the trend value exceeds the predetermined threshold, the tissue change estimation function 34c estimates that the tissue is soft. On the other hand, if the trend value is less than or equal to the predetermined threshold, the tissue change estimation function 34c estimates that the tissue is hard. While the present exemplary embodiment describes the example where an estimation of whether the tissue is hard or soft is made by comparing the trend value with the threshold, the present exemplary embodiment is not limited thereto. For example, the tissue change estimation function 34c may estimate whether the tissue is hard or soft by inputting the trend value to a trained model that accepts a trend value as input and outputs an estimation of whether the tissue is hard or soft.

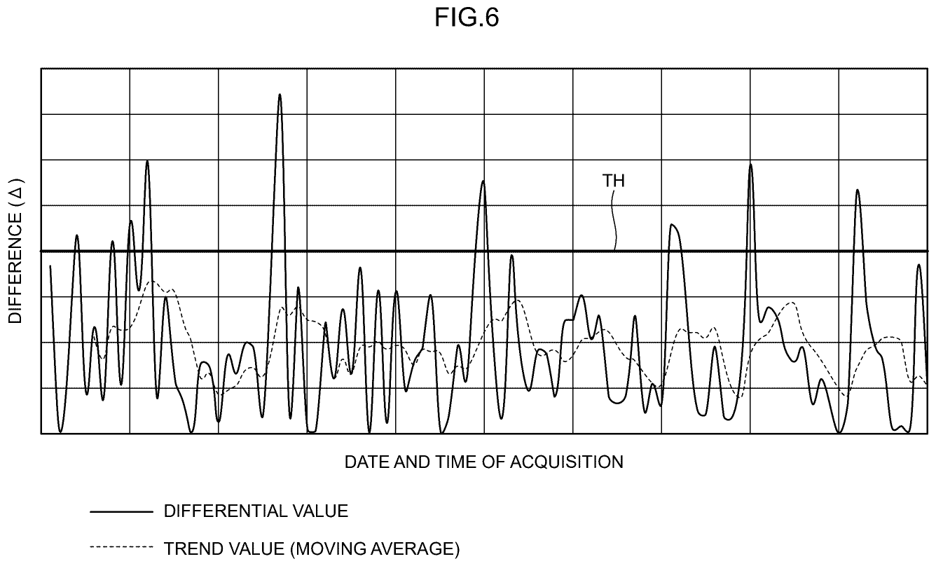

FIG. 6 is a graph illustrating an example of the difference Δ relative to the acquisition date and time, and the trend value. The vertical axis illustrated in FIG. 6 represents the magnitude of the difference Δ. The horizontal axis illustrated in FIG. 6 represents the acquisition date and time (e.g., in the order of seconds). As illustrated in FIG. 6, the fluctuation in the trend value is smaller than the fluctuation in the difference value. As illustrated in FIG. 6, if the tissue surrounding the intracorporeal device 10 is soft, or if a large amount of liquid such as fluid is present in the vicinity of the tissue, the trend value increases (the difference between the devices increases).

On the other hand, if the tissue surrounding the intracorporeal device 10 is hard, or if a small amount of liquid such as fluid is present in the vicinity of the tissue, the trend value decreases. A horizontal line TH illustrated in FIG. 6 represents the predetermined threshold. In this case, if the trend value exceeds the predetermined threshold, the tissue change estimation function 34c estimates that the tissue is soft.

For example, the tissue surrounding a tumor tissue becomes softer as the tumor tissue becomes smaller (more cancer cells are killed) by chemotherapy, radiation therapy, or the like. In this case, the mobility of the intracorporeal device 10 increases as compared to a case where the size of a tumor is large. The trend value decreases as the mobility of the intracorporeal device 10 increases. Accordingly, in this case, the tissue change estimation function 34c uses the trend value to determine the therapeutic effect on the tumor as a state corresponding to the disorder in the tissue.

On the other hand, in a case where a benign tumor changes into a malignant tumor (becomes cancerous), the surrounding tissue becomes hard due to enlargement of the cancer. In this case, the intracorporeal device 10 becomes difficult to move compared to a benign tumor. Since the trend value increases as the intracorporeal device 10 becomes difficult to move, the tissue change estimation function 34c uses the trend value to determine a degree of malignancy of a tumor or the like as a state corresponding to the disorder in the tissue.

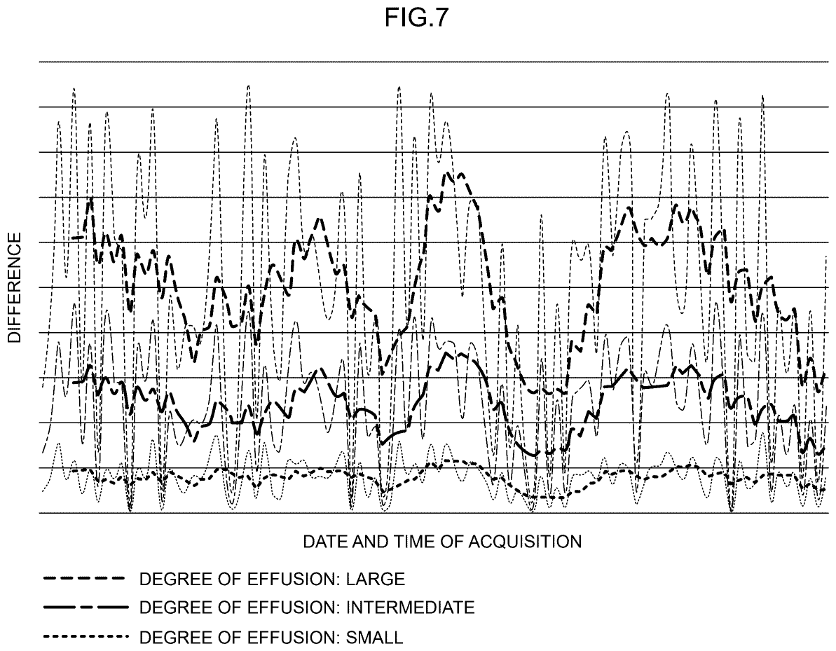

FIG. 7 is a graph illustrating an example of the difference Δ in a case where the disease condition of the subject P is pleural effusion, and the trend value. As illustrated in FIG. 7, tendencies of the difference and the trend value vary depending on a degree of pleural effusion. Accordingly, the tissue change estimation function 34c appropriately sets a threshold TH and compares the trend value with the threshold TH, thereby making it possible to estimate the degree of pleural effusion.

Specifically, the tissue change estimation function 34c further estimates the state (disease condition) corresponding to the disorder in the tissue based on the estimation result of the change in the characteristics of the tissue (result of comparison between the threshold TH and the trend value) or the like. In the example illustrated in FIG. 7, the tissue change estimation function 34c estimates the degree of pleural effusion based on the result of comparison between the threshold TH and the trend value.

The degree of pleural effusion indicating the state corresponding to the disorder in the tissue is not limited to a plurality of thresholds for the degree of accumulation, e.g., “large”, “medium”, and “small”, as illustrated in FIG. 7. For example, the tissue change estimation function 34c may calculate a ratio of a current trend value based on a reference value, which corresponds to a trend value obtained at a timing when the disease condition of the subject P has previously deteriorated, as a pleural effusion accumulation index indicating the condition of pleural effusion. Further, the tissue change estimation function 34c may calculate a difference between the previous pleural effusion accumulation index and the currently calculated pleural effusion accumulation index.

The display control function 34d of the processing circuitry 34 causes the terminal 40 to display the estimation result of the change in the characteristics of the tissue. Further, the display control function 34d causes the terminal 40 to display, for example, the estimated state of the disorder. Specifically, the display control function 34d causes the display 41 of the terminal 40 to display the estimation result of the change in the characteristics of the tissue and/or the estimated state of the disorder. The display control function 34d may cause the display 32 to display the estimation result of the change in the characteristics of the tissue and/or the estimated state of the disorder.

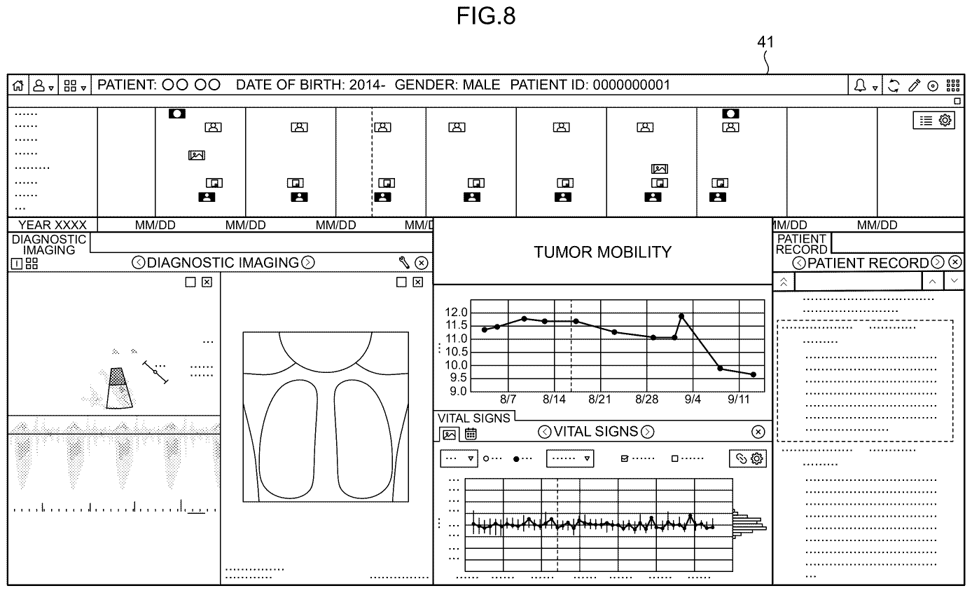

FIG. 8 is a diagram illustrating an example of an estimation result displayed on the display 41 of the terminal 40. The terminal 40 illustrated in FIG. 8 corresponds to a terminal for a medical worker, such as a client apparatus and/or a viewer apparatus in an electronic health record system. FIG. 8 illustrates the mobility of a tumor as an example of the estimation result. In this case, the tissue change estimation function 34c may estimate the size of the tumor based on the previous estimation result and the current estimation result.

For example, the tissue change estimation function 34c estimates the size of a tumor (the degree of malignancy or the like) based on a correlation between the size of the tumor and the mobility of the tumor analyzed in advance. Specifically, in a case where the intracorporeal device 10 is implanted in tumor tissue, since the tumor tissue becomes difficult to move as the trend value decreases, the tissue change estimation function 34c estimates that the size of the tumor has increased.

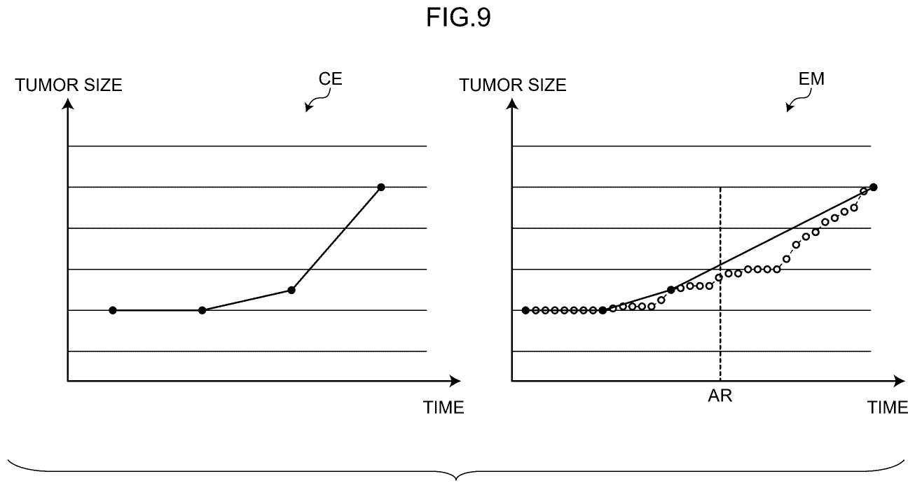

FIG. 9 is a diagram illustrates examples of a graph CE indicating the size of a tumor over time according to a comparative example, and a graph EM indicating the size of a tumor over time according to the present exemplary embodiment. As illustrated in FIG. 9, in the present exemplary embodiment, the size of the tumor can be displayed on the terminal 40 at a point AR before the tumor becomes large as compared to the comparative example.

Specifically, in the graph CE according to the comparative example in which the size of the tumor estimated based on an imaging test (radiography, X-ray CT, or the like) is displayed, there is a limitation on the frequency of the imaging test due to exposure to radiation or the like. Accordingly, it may be difficult to detect, for example, enlargement of a tumor at an early stage. On the other hand, in the graph EM according to the present exemplary embodiment, the trend value can be acquired more frequently than in the comparative example, so that the user can detect a sign of a change in the size of the tumor at an earlier stage than in the comparative example.

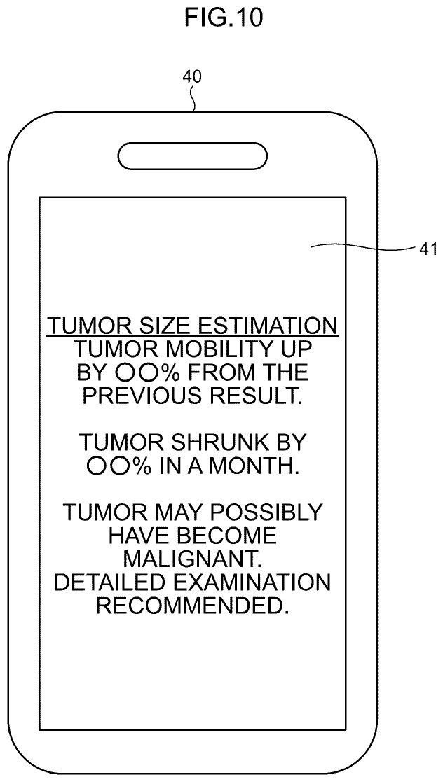

FIG. 10 is a diagram illustrating an example of an estimation result regarding a tumor displayed on the display 41 of the terminal 40. The display 41 illustrated in FIG. 10 corresponds to a display of the terminal 40 for a medical worker such as a doctor in charge. The estimation result illustrated in FIG. 10 indicates, for example, the ratio between the trend value in the previous estimation result and the trend value in the current estimation result obtained by the tissue change estimation function 34c, the ratio between the size of the tumor in the previous estimation result and the size of the tumor in the current estimation result, and a recommendation of a detailed examination as a result of estimation of malignancy based on the difference between the hardness of the tumor in the previous estimation result and the hardness of the tumor in the current estimation result. The display control function 34d causes the display 41 to display the ratios and the recommendation of the detailed examination described above.

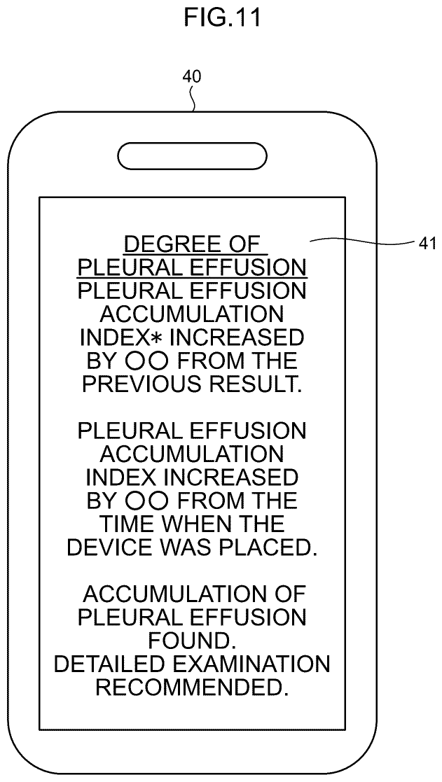

FIG. 11 is a diagram illustrating an example of an estimation result regarding pleural effusion displayed on the display 41 of the terminal 40. The display 41 illustrated in FIG. 11 corresponds to a display of the terminal 40 for a medical worker such as a doctor in charge, like in FIG. 10. The pleural effusion accumulation index indicating the estimation result illustrated in FIG. 11 is estimated by the tissue change estimation function 34c, for example, based on the trend value in the previous estimation result and the trend value in the current estimation result.

Further, the tissue change estimation function 34c may calculate a difference from the pleural effusion accumulation index corresponding to the current trend value based on the trend value obtained at a point when the intracorporeal device 10 is placed. In this case, the display control function 34d may cause the display 41 to display the pleural effusion accumulation index corresponding to the difference as illustrated in FIG. 11. In addition, as illustrated in FIG. 11, the display control function 34d causes the display 41 to display, for example, a recommendation of a detailed examination, based on the difference.

The overall configuration of the biological tissue information processing system 1 according to the exemplary embodiment has been described above. Processing in which the biological tissue information processing apparatus 30 estimates a change in characteristics of tissue and causes the terminal 40 to display an estimation result (this processing is hereinafter referred to as characteristic change estimation processing) will be described below.

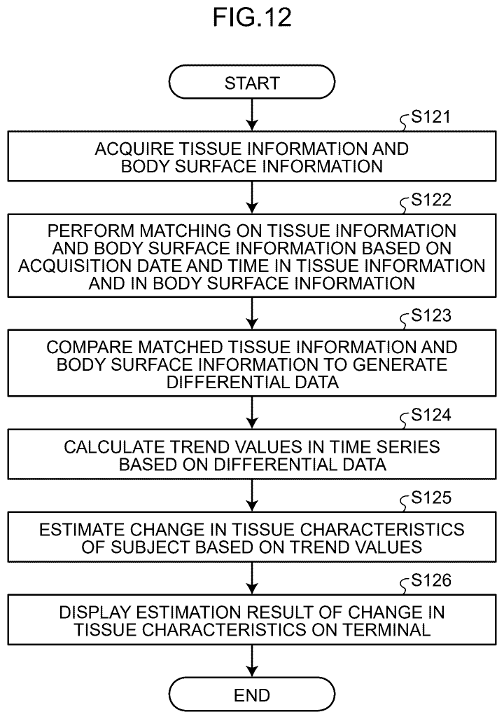

FIG. 12 is a flowchart illustrating an example of a procedure of characteristic change estimation processing. Assume that, prior to execution of the characteristic change estimation processing, the intracorporeal device 10 is preliminarily located in tissue related to the disease condition of the subject P as illustrated in FIG. 2. Also, assume that, prior to execution of the characteristic change estimation processing, the extracorporeal device 20 is preliminarily attached to the body surface of the subject P in the vicinity of the tissue in which the intracorporeal device 10 is located as illustrated in FIG. 2.

### (Characteristic Change Estimation Processing)

### (Step S121)

The processing circuitry 34 causes the acquisition function 34a to acquire the tissue information TI indicating a state of tissue related to a disease in the body of the subject P, and the body surface information BS indicating a state of a body surface of the subject P. For example, the acquisition function 34a acquires the tissue information TI including an acceleration related to the tissue in the body of the subject P and an acquisition date and time of the acceleration from the intracorporeal device 10. Further, the acquisition function 34a acquires the body surface information BS including an acceleration on the body surface of the subject P and an acquisition date and time of the acceleration from the extracorporeal device 20. The acquisition function 34a causes the memory 33 to store the acquired tissue information TI and the acquired body surface information BS.

### (Step S122)

The processing circuitry 34 causes the comparison function 34b to perform matching on the tissue information TI and the body surface information BS based on the acquisition date and time in the tissue information TI and the acquisition date and time in the body surface information BS. Specifically, the comparison function 34b generates the matching result BR as illustrated in FIG. 4 by performing matching on the tissue information TI and the body surface information BS using the acquisition dates and times. The comparison function 34b causes the memory 33 to store the matching result BR.

### (Step S123)

The processing circuitry 34 causes the comparison function 34b to compare the matched tissue information TI and the body surface information BS and to generate differential data including a plurality of differences A for respective acquisition dates and times. For example, the comparison function 34b generates the differential data by applying the acceleration in the tissue information TI and the acceleration in the body surface information BS to Formula (1) or Formula (2) for each of the plurality of acquisition dates and times. The comparison function 34b causes the memory 33 to store the generated differential data.

### (Step S124)

The processing circuitry 34 causes the tissue change estimation function 34c to calculate trend values in time series based on the differential data. Specifically, the tissue change estimation function 34c calculates a plurality of trend values corresponding to the plurality of acquisition dates and times, respectively, by applying a moving average to each of the acquisition dates and times in the differential data. The tissue change estimation function 34c causes the memory 33 to store the plurality of generated trend values.

### (Step S125)

The processing circuitry 34 causes the tissue change estimation function 34c to estimate a change in characteristics of the tissue in the subject P based on the plurality of trend values. For example, the tissue change estimation function 34c compares each of the plurality of trend values with a designated threshold, thereby estimating the change in the tissue. The tissue change estimation function 34c further estimates the state (disease condition) corresponding to the disorder in the tissue in the subject P based on the estimation result of the change in the characteristics of the tissue and the like.

For example, the tissue change estimation function 34c estimates the disease condition of the subject P based on the current trend value and the previous trend value such as the trend value at a timing when the disease condition of the subject P has deteriorated. Specifically, the tissue change estimation function 34c estimates the disease condition of the subject P using a correspondence table of the disease condition of the disorder corresponding to the difference or ratio between the previous trend value and the current trend value. The tissue change estimation function 34c causes the memory 33 to store the estimation result including various estimations as described above.

### (Step S126)

In the processing circuitry 34, the display control function 34d causes the display 41 of the terminal 40 to display the estimation result. Further, the display control function 34d causes the display 41 to display the estimated state of the disorder. The display control function 34d may cause the display 32 of the biological tissue information processing apparatus 30 to display the estimation result.

For example, the display control function 34d causes the display 41 in the terminal 40 of a display system (an electronic health record system, an integrated viewer, or the like) for a medical worker to display the estimation result. Further, the display control function 34d causes the terminal 40 to display the estimation result on a health support application, a sleep analysis application, or the like installed on the terminal 40 owned by the subject P.

As a modified example of the present exemplary embodiment, in a case where the disease condition of the subject P is pleural effusion, the acquisition function 34a may acquire the tissue information TI and the body surface information BS while the subject P is engaging in intense physical activity. In this case, the tissue change estimation function 34c determines the degree of accumulation of fluid in the subject P by detecting that the intracorporeal device 10 is not moving to a large extent while the heart of the subject P is expected to be moving vigorously. Further, the acquisition function 34a may acquire the tissue information TI and the body surface information BS while the subject P is not engaging in intense physical activity. In this case, the tissue change estimation function 34c determines the degree of accumulation of fluid in the subject P by detecting that the intracorporeal device 10 is moving while the heart of the subject P is expected to be moving vigorously.

The biological tissue information processing apparatus 30 according to the exemplary embodiment described above acquires the tissue information TI indicating a state of tissue related to a disease in the body of the subject P, and the body surface information BS indicating a state of a body surface of the subject P, compares the tissue information TI with the body surface information BS, calculates a result of comparison between the tissue information TI and the body surface information BS, estimates a change in characteristics of the tissue based on a temporal change of the comparison result, and causes the terminal 40 to display the estimation result of the change in the characteristics of the tissue. In the biological tissue information processing apparatus 30 according to the exemplary embodiment, the characteristics of the tissue is the hardness of tissue. Further, the biological tissue information processing apparatus 30 according to the exemplary embodiment acquires the tissue information TI from the intracorporeal device 10 located in an area associated with the tissue of the subject P, and acquires the body surface information BS from the extracorporeal device 20 located outside the body of the subject P, or acquires the body surface information BS by inputting the tissue information TI to a trained model.

In the biological tissue information processing apparatus 30 according to the exemplary embodiment, the tissue information TI and the body surface information BS include an acquisition date and time of the tissue information TI and an acquisition date and time of the body surface information BS, respectively. The biological tissue information processing apparatus 30 according to the exemplary embodiment performs matching on the acquisition date and time of the tissue information TI and the acquisition date and time of the body surface information BS, and calculates the difference between the tissue information TI and the body surface information BS for each acquisition date and time as a comparison result using the matched tissue information TI and the body surface information BS.

Accordingly, in the biological tissue information processing apparatus 30 according to the exemplary embodiment, it is possible to acquire a difference between the degree of motion (acceleration) of the intracorporeal device 10 and the degree of motion (acceleration) of the extracorporeal device 20 and a temporal change in the difference simply and frequently without performing an image diagnosis or the like on the subject P, thereby making it possible to estimate a change in characteristics of the tissue surrounding the intracorporeal device 10. Consequently, the biological tissue information processing apparatus 30 according to the exemplary embodiment can detect a change in characteristics of tissue in a lesional region of the subject P at an early stage and present the estimation result to the subject P, a medical worker (user) such as a doctor in charge, or the like. With the biological tissue information processing apparatus 30 according to the exemplary embodiment, the user can detect a sign of a change in the size of a tumor at an earlier stage than in the graph CE according to the comparative example, for example, as illustrated in FIGS. 7 and 9.

In addition, the biological tissue information processing apparatus 30 according to the exemplary embodiment further estimates a state corresponding to a disorder in the tissue of the subject P based on the estimation result, and further causes the terminal 40 to display the estimated state of the disorder. Thus, the biological tissue information processing apparatus 30 according to the exemplary embodiment can present the change in the characteristics of the tissue in the lesional region in the subject P to the user, and also present measures based on the estimated change in the disease condition to the user, for example, as illustrated in FIGS. 10 and 11.

As described above, the biological tissue information processing apparatus 30 according to the exemplary embodiment can detect a change in characteristics of tissue related to a disease at an early stage, which makes it possible to take necessary measures for a lesion at an early stage and improve the QOL of the subject P.

### First Application Example

In a first application example, body motion information indicating a body motion of the subject P is acquired from a body motion detection device for detecting a body motion of the subject P, and tissue information is corrected. Examples of the body motion detection device include an electrocardiogram monitor, a respirometer, and a pulse rate meter. The processing circuitry 34 causes the acquisition function 34a to acquire the body motion information from the body motion detection device. The body motion information includes body motion data indicating the body motion of the subject P, and an acquisition date and time when the body motion data is acquired. The acquisition function 34a causes the memory 33 to store the body motion information.

The processing circuitry 34 causes the comparison function 34b to correct the tissue information TI using the acquired body motion information. For example, the comparison function 34b corrects the acceleration on the tissue (tissue information) using the tissue information TI and the body motion information having the same acquisition date and time. In the corrected acceleration, noise due to the body motion of the subject P is reduced. In other words, an effect of noise due to the body motion of the subject P is reduced in the corrected tissue information. The comparison function 34b compares the corrected tissue information with the body surface information and calculates a comparison result.

The biological tissue information processing apparatus 30 according to the first application example corrects the tissue information based on the body motion information indicating the body motion of the subject P, thereby making it possible to improve accuracy of the trend value. Accordingly, the biological tissue information processing apparatus 30 according to the first application example can improve accuracy of the estimation result and can present the estimation result to the user. Therefore, the biological tissue information processing apparatus 30 according to the first application example enables the user to detect a change in characteristics of tissue caused by a disease at an early stage, thereby making it possible to take necessary measures for a lesion with higher accuracy. As described above, the biological tissue information processing apparatus 30 according to the first application example can further improve the QOL of the subject P. Other effects are similar to those of the exemplary embodiment, and thus descriptions thereof are omitted.

### Second Application Example

In a second application example, body motion information is acquired from a body motion detection device, and the tissue information TI and the body surface information BS are acquired at a predetermined timing when the body motion becomes small based on the body motion information. Hereinafter, for the purpose of providing a concrete description, the body motion detection device will be described as an electrocardiogram monitor. In this case, the body motion information corresponds to an electrocardiographic waveform. The body motion detection device may also function as the extracorporeal device 20.

The processing circuitry 34 causes the acquisition function 34a to acquire an electrocardiographic waveform of the subject P from the electrocardiogram monitor. The acquisition function 34a acquires the tissue information TI and the body surface information BS during a period (hereinafter referred to as a body motion minimum period) in which the body motion of the subject P is smaller than that in any other period in the electrocardiographic waveform. The body motion minimum period is, for example, a predetermined period including an end-diastolic phase and/or a predetermined period including an end-systolic phase, and corresponds to the predetermined timing. In this case, the acquisition function 34a acquires the tissue information TI and the body surface information BS at the same timing each time. In a case where the body motion detection device is a pulse rate meter or a blood pressure monitor, at a set value, the acquisition function 34a acquires the tissue information TI and the body surface information BS at the same timing each time.

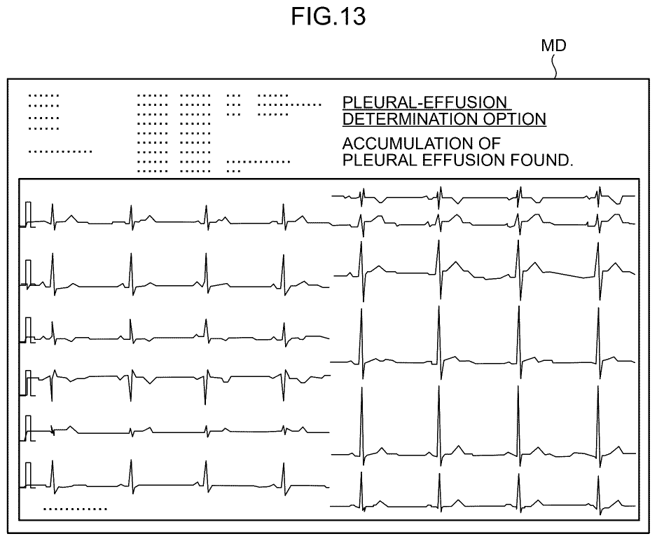

The processing circuitry 34 may cause the display control function 34d to display an estimation result estimated by the tissue change estimation function 34c and the like on a monitor of the body motion detection device. FIG. 13 is a diagram illustrating an example where the estimation result is displayed on a display MD of an electrocardiogram monitor in a case where the electrocardiogram monitor is used as the body motion detection device. As illustrated in FIG. 13, the user can check the estimation result displayed by the display control function 34d, as well as the electrocardiographic waveform, on the electrocardiogram of the subject P.

The biological tissue information processing apparatus 30 according to the second application example can acquire the tissue information TI and the body surface information BS at a predetermined timing when the body motion becomes small (body motion minimum period) using the body motion information indicating the body motion of the subject P. Consequently, the biological tissue information processing apparatus 30 according to the second application example can reduce noise due to the body motion of the subject P, which leads to an improvement in the accuracy of the trend value. Other effects are similar to those of the exemplary embodiment and the like, and thus descriptions thereof are omitted.

In a case where the technical idea of the present exemplary embodiment is implemented as a biological tissue information processing method, the biological tissue information processing method includes acquiring tissue information indicating a state of tissue related to a disease in the body of the subject P, and body surface information indicating a state of a body surface of the subject P, comparing the acquired tissue information with the acquired body surface information, calculating a result of comparison between the acquired tissue information and the acquired body surface information, estimating a change in characteristics of the tissue based on a temporal change of the calculated comparison result, and causing the terminal 40 to display an estimation result of a change in characteristics of the tissue. A procedure for characteristic change estimation processing to be implemented by the biological tissue information processing method is compliant with the exemplary embodiment and the like. Effects of the biological tissue information processing method are similar to those of the exemplary embodiment. Thus, descriptions of the processing procedure and effects of the characteristic change estimation processing according to the biological tissue information processing method are omitted.

In a case where the technical idea of the present exemplary embodiment is implemented as a biological tissue information processing program, the biological tissue information processing program causes a computer to execute the biological tissue information processing method including acquiring tissue information indicating a state of tissue related to a disease in the body of the subject P, and body surface information indicating a state of a body surface of the subject P, comparing the acquired tissue information with the acquired body surface information, calculating a result of comparison between the acquired tissue information and the acquired body surface information, estimating a change in characteristics of the tissue based on a temporal change of the calculated comparison result, and causing the terminal 40 to display an estimation result of a change in characteristics of the tissue. For example, the characteristic change estimation processing can be implemented by installing the biological tissue information processing program on a computer such as the biological tissue information processing apparatus 30 illustrated in FIG. 1 and loading the biological tissue information processing program into a memory. In this case, the program for causing the computer to execute the processing can be stored in a storage medium such as a magnetic disk (e.g., a hard disk), an optical disk (e.g., a compact disc (CD)-read-only memory (ROM), a digital versatile disc (DVD), or the like), or a semiconductor memory, and can be distributed.

The distribution of the biological tissue information processing program is not limited to the case where the above-described media is used. For example, the program may be distributed using an electric communication function such as downloading via the Internet. The processing procedure in the biological tissue information processing program is compliant with the characteristic change estimation processing. Effects of the biological tissue information processing program are similar to those of the exemplary embodiment. Thus, descriptions of the processing procedure and effects of the characteristic change estimation processing according to the biological tissue information processing program are omitted.

According to at least the exemplary embodiments, modified examples, application examples, and the like described above, it is possible to detect a change in characteristics of tissue related to a disease in the body of the subject P at an early stage. Consequently, the QOL and the like of the subject P can be improved.

While certain embodiments have been described, these embodiments have been presented by manner of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A biological tissue information processing apparatus comprising processing circuitry configured to:
acquire tissue information indicating a state of tissue related to a disease in a body of a subject, and body surface information indicating a state of a body surface of the subject;
compare the tissue information with the body surface information and calculate a result of a comparison between the tissue information and the body surface information;
estimate a change in characteristics of the tissue based on a temporal change of the result of the comparison; and
control a terminal to display an estimation result of the change in the characteristics of the tissue.

2. The biological tissue information processing apparatus according to claim 1, wherein the characteristics include hardness of the tissue and accumulation of fluid or air around the tissue.

3. The biological tissue information processing apparatus according to claim 1,
wherein the tissue information and the body surface information include an acquisition date and time of the tissue information and an acquisition date and time of the body surface information, respectively, and
wherein the processing circuitry is further configured to:
perform matching on the acquisition date and time of the tissue information with the acquisition date and time of the body surface information; and
calculate, as the result of the comparison, a difference between the tissue information and the body surface information for each of the acquisition dates and times using the matched tissue information and the body surface information.

4. The biological tissue information processing apparatus according to claim 1, wherein the processing circuitry is further configured to:
acquire the tissue information from an intracorporeal device located in an area associated with the tissue; and
acquire the body surface information from an extracorporeal device located outside the body of the subject, or acquire the body surface information by inputting the tissue information to a trained model.

5. The biological tissue information processing apparatus according to claim 1, wherein the processing circuitry is further configured to:
acquire body motion information indicating a body motion of the subject from a body motion detection device configured to detect a body motion of the subject;
correct the tissue information using the body motion information; and
compare the corrected tissue information with the body surface information and calculate the result of the comparison.

6. The biological tissue information processing apparatus according to claim 1, wherein the processing circuitry is further configured to:
estimate a state corresponding to a disorder in the tissue based on the estimation result; and
control the terminal to display the state of the disorder.

7. The biological tissue information processing apparatus according to claim 1, wherein the processing circuitry is further configured to:
acquire body motion information indicating a body motion of the subject from a body motion detection device configured to detect a body motion of the subject; and
acquire the tissue information and the body surface information at a predetermined timing when the body motion becomes small based on the body motion information.

8. A biological tissue information processing method comprising:
acquiring tissue information indicating a state of tissue related to a disease in a body of a subject, and body surface information indicating a state of a body surface of the subject;
comparing the tissue information with the body surface information and calculating a result of a comparison between the tissue information and the body surface information;
estimating a change in characteristics of the tissue based on a temporal change of the result of the comparison; and
controlling a terminal to display an estimation result of the change in the characteristics of the tissue.

9. A non-transitory computer-readable storage medium storing a biological tissue information processing program for causing a computer to:
acquire tissue information indicating a state of tissue related to a disease in a body of a subject, and body surface information indicating a state of a body surface of the subject;
compare the tissue information with the body surface information and calculate a result of a comparison between the tissue information and the body surface information;
estimate a change in characteristics of the tissue based on a temporal change of the result of the comparison; and
control a terminal to display an estimation result of the change in the characteristics of the tissue.
